(19) United States
(12) Patent Application Publication
(10) Pub. No.: US 2023/0075925 A1
(43) Pub. Date: Mar. 9, 2023

(54) NON-PERIPHERAL QUATERNARY AMMONIUM-MODIFIED ZINC PHTHALOCYANINE AND PREPARATION METHOD AND USE THEREOF

(71) Applicant: FUZHOU UNIVERSITY, Fujian (CN)

(72) Inventors: Jiandong HUANG, Fujian (CN); Biyuan ZHENG, Fujian (CN); Meirong KE, Fujian (CN)

(21) Appl. No.: 17/793,297

(22) PCT Filed: Jan. 15, 2021
(86) PCT No.: PCT/CN2021/072117

§ 371 Date: Jul. 15, 2022

(30) Foreign Application Priority Data
Jan. 16, 2020 (CN) 202010048304.0

(51) Int. Cl. C07D 487/22; A61K 41/00; A61K 31/555; A61K 39/395; A61P 35/00; A61P 17/00

(52) U.S. Cl. CPC C07D 487/22; A61K 41/0071; A61K 31/555; A61K 39/3955; A61P 35/00; A61P 17/00

(57) ABSTRACT

The present disclosure provides a non-peripheral quaternary ammonium-modified zinc phthalocyanine and a preparation method and use thereof, belonging to the field of preparation of photodynamic drugs or photosensitizers. The non-peripheral quaternary ammonium-modified zinc phthalocyanine can be used as a photosensitizer for a photodynamic therapy (PDT) and photodynamic diagnosis, and can also be used for a photodynamic-immune synergistic therapy. Due to a unique structure, the non-peripheral quaternary ammonium-modified zinc phthalocyanine can be combined with an immune checkpoint blocker to achieve an excellent synergistic anti-tumor effect, which has a significant prospect for use in treating a metastatic tumor.

### CROSS REFERENCE TO RELATED APPLICATION

The present application claims priority of Chinese Patent Application No. CN202010048304.0 filed with the China National Intellectual Property Administration (CNIPA) on Thursday, Jan. 16, 2020 and entitled “NON-PERIPHERAL QUATERNARY AMMONIUM-MODIFIED ZINC PHTHALOCYANINE AND PREPARATION METHOD AND USE THEREOF”, which is incorporated herein by reference in its entirety.

### TECHNICAL FIELD

The present disclosure relates to the technical field of photodynamic drugs and photosensitizers, in particular to a non-peripheral quaternary ammonium-modified zinc phthalocyanine and a preparation method and use thereof.

### BACKGROUND ART

Phthalocyanine complexes are an important class of functional materials, which can be developed into functional materials for different purposes through different structural modifications. By introducing suitable substituents and central ions on a phthalocyanine ring, it is possible to develop oxidation catalysts, desulfurization catalysts, nonlinear optical materials, photosensitive drugs, liquid crystal materials, optical recording materials, or photoconductive materials. However, it requires creative work to control the substituents and the central ions to obtain target functional compounds.

The phthalocyanine complexes as photosensitizers have an eye-catching prospect for use in the photodynamic therapy (PDT). The PDT is essentially use of photosensitization of the photosensitizers (also known as the photosensitive drugs) in the medical field. An action process of the PDT includes: injecting a photosensitizer into the body; after a period of time (this period of time is to allow the drug to be relatively enriched in the target body), irradiating the target body with light of a specific wavelength (for a target in the body cavity, a light source can be introduced by means of an interventional technology such as optical fibers); the photosensitizers enriched in the target body, under the excitation of light, inspiring a series of photophysical and photochemical reactions to generate reactive oxygen species, thereby destroying the target body (such as cancer cells and cancer tissues).

In some developed countries, PDT has become the fourth routine method of cancer treatment. Compared with traditional therapies, such as surgery, chemotherapy, and radiation therapy, the PDT has attracted much attention due to the greatest advantage of selective destruction on the cancer tissues with no surgery requirement and less side effects.

In addition, recent studies have also shown that PDT can further effectively treat non-cancer diseases such as bacterial infections, oral diseases, macular degeneration-caused eye diseases, arteriosclerosis, traumatic infections, and skin diseases. Photosensitizers can further be used for photodynamic disinfection, most notably for the sterilization of water, blood and blood derivatives. Meanwhile, due to fluorescence properties, the photosensitizers are also used for photodynamic diagnosis.

A key to PDT is the photosensitizers. The photosensitizers currently approved for official clinical use are mainly hematoporphyrin derivatives. In the United States, Canada, Germany, Japan and other countries, Photofrin is used (the Food and Drug Administration (FDA) officially approved Photofrin for clinical treatment of cancers in 1995). The Photofrin is a mixture of hematoporphyrin oligomers extracted from cow blood and chemically modified. Hematoporphyrin derivatives have shown a certain curative effect, but also exposed serious shortcomings: a maximum absorption wavelength (380 nm to 420 nm) is not in a red light region (650 nm to 800 nm) that has better transmittance to human tissues. Moreover, the hematoporphyrin derivatives are mixtures with an unstable composition and have high skin phototoxicity, which has limited the clinical uses. Therefore, the development of a next-generation photodynamic drug (photosensitizer) has become an international research hotspot.

The phthalocyanine complexes as photosensitizers have attracted attention due to a maximum absorption wavelength in the red light region that easily penetrates human tissues and a strong photosensitization ability. Among various phthalocyanine complexes, the zinc phthalocyanine as a novel photosensitizer is highly valued for the following reasons: (1) the zinc phthalocyanine can introduce hydrophilic groups in a peripheral ring, to more effectively prevent the aggregation of phthalocyanine rings and ensure the exertion of a photosensitization ability of phthalocyanine; and (2) zinc has relatively high biocompatibility and no dark toxicity. Peripheral asymmetric tetra-substituted zinc phthalocyanine (ZnPcS₂P₂K₂) developed by Fuzhou University in China shows a relatively high photodynamic activity and has entered phase II clinical trials. However, the ZnPcS₂P₂K₂ has a complex synthetic route and high preparation cost, and has isomers. Therefore, there is an urgent need to select new zinc phthalocyanine photosensitizers with high photosensitivity, simple preparation, low cost, and no isomers. In addition, it is also a problem to be overcome at present that the photosensitizers (including phthalocyanine photosensitizers) currently in clinical trials are ineffective for deep tumors and metastatic tumors.

In recent years, cancer immunotherapy based on mobilizing and awakening the human immune system to eliminate cancer cells has received great attention. In particular, immunotherapy based on immune checkpoint inhibitors (also known as immune checkpoint blockade, ICB) has made breakthrough progress, and related basic researches thereof won the 2018 Nobel Prize. Currently, one PD-L1 antibody (anezolizumab) and two PD-1 antibodies (nivolumab and pembrolizumab) have been approved by the FDA for the treatment of advanced melanoma, non-small cell lung cancer, and bladder cancer. Among them, the nivolumab was also approved by the China Food and Drug Administration (CFDA) in June 2018 for the treatment of non-small cell lung cancer. However, since ICB relies on tumors with high PD-L1 expression or pre-existing infiltrating T cells capable of expressing the PD-1, PD-1/PD-L1 antibodies are only about 20% effective in non-selective people with advanced solid tumors, are generally accompanied by autoimmune allergic side effects, and has a high cost during treatment. Therefore, it is a main challenge to be solved in clinical promotion to expand the applicability of ICB.

The combined use of PDT and ICB is expected to overcome the respective problems of PDT and ICB, showing potential advantages: it is expected to cooperate with ICB to activate the human immune system to inhibit the growth and metastasis of distal tumors, sensitizing some tumor types that are originally insensitive to ICB therapy; in addition, the dosage of immune checkpoint blockers can also be reduced, thereby greatly reducing the cost of treatment and relieving the side effects of immune allergy.

However, there is still a lack of effective combined photosensitizers, and no photosensitizers of high efficiency and synergy with ICB have been approved for clinical uses. There is also no report on the combination of phthalocyanine photosensitizers with the PD-L1 antibody. In particular, the structural characteristics of photosensitizers that can be used in combination with ICB therapy to achieve a highly synergistic anti-metastatic effect have not been thoroughly studied. Therefore, it is of great value to develop a phthalocyanine photosensitizer that can be used in highly synergistic combination with ICB.

### SUMMARY

An objective of the present disclosure is to provide a non-peripheral quaternary ammonium-modified zinc phthalocyanine and a preparation method and use thereof. The zinc phthalocyanine shows a high photodynamic activity, and more importantly can cooperate well with an immune checkpoint blocker PD-L1 to exert a highly effective anti-tumor function.

To achieve the above objective, the present disclosure adopts the following technical solutions.

The present disclosure provides a non-peripheral quaternary ammonium-modified zinc phthalocyanine, having a structural formula shown as follows:

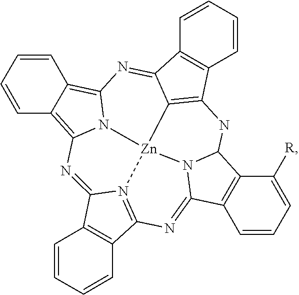

the above formula represents a non-peripheral monosubstituted zinc phthalocyanine complex. Zinc phthalocyanine is a phthalocyanine complex whose central ions are zinc ions; phthalocyanine is an abbreviation of tetrabenzotetraazaporphyrin. Substituents are located in an a position of a phthalocyanine ring, called a non-peripheral position, and a substituent R is selected from the following group:

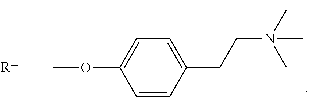

The present disclosure further provides a preparation method of the non-peripheral quaternary ammonium-modified zinc phthalocyanine, including the following steps:

(1) taking 1-[4-(aminoethyl)phenoxy] zinc phthalocyanine and methyl iodide as reactants, at a ratio that 1 mg of the 1-[4-(aminoethyl)phenoxy] zinc phthalocyanine requires 50 mg to 200 mg of the methyl iodide;

(2) conducting a reaction on 1 mg of the 1-[4-(aminoethyl)phenoxy] zinc phthalocyanine and the methyl iodide in 0.3 mL to 3 mL of N,N-dimethylformamide as a solvent under the protection of nitrogen at 0° C. to a room temperature for 5 h to 50 h; and removing excess raw materials and impurities by solvent cleaning and column chromatography isolation to obtain 1-[4-(N,N,N-trimethyl-2-aminoethyl)phenoxy] zinc phthalocyanine iodide, namely the non-peripheral quaternary ammonium-modified zinc phthalocyanine.

The present disclosure further provides use of the non-peripheral quaternary ammonium-modified zinc phthalocyanine in preparation of a photodynamic drug, or a photosensitizer, or a photodynamic-immune combined photosensitizer. The photosensitizer is also known as a photosensitive drug, or a photosensitive pharmaceutical preparation, or a photodynamic agent in the field of biomedicine. The photodynamic drug or the photosensitizer may be used for a PDT, photodynamic diagnosis, or photodynamic disinfection. The PDT may include a PDT of a malignant tumor, a PDT of a benign tumor, an in vitro photodynamic purification therapy of leukemia, or a PDT of a non-cancer disease. The non-cancer disease may include a bacterial infection, an oral disease, a macular degeneration-caused eye disease, arteriosclerosis, a traumatic infection, a skin disease, or a viral infection. The photodynamic disinfection may include photodynamic sterilization and purification of blood or a blood derivative, photodynamic sterilization and disinfection of water, and photodynamic disinfection of a medical or household appliance.

A preparation method of the photodynamic drug or the photosensitizer includes the following steps: dissolving the non-peripheral quaternary ammonium-modified zinc phthalocyanine using water or a mixed solution of water and other substances as a solvent, to obtain a certain concentration of photosensitizer, where the zinc phthalocyanine has a concentration of not more than a saturation concentration thereof, and the other substances have a mass fraction of not higher than 10%; in the photosensitizer and the photodynamic drug, the non-peripheral quaternary ammonium-modified zinc phthalocyanine has a concentration of independently 0.1 mM or 0.2 mM; an antioxidant, a buffer and an isotonic agent are added as additives to the prepared solution to maintain a chemical stability and biocompatibility of the photosensitizer; the other substances are one or a mixture of more selected from the group consisting of a castor oil derivative (Cremophor EL), dimethyl sulfoxide (DMSO), ethanol, glycerol, N,N-dimethylformamide, polyethylene glycol 300 to polyethylene glycol 3000, cyclodextrin, glucose, Tween, and polyethylene glycol monostearate.

The present disclosure further provides a combination therapy method of tumor-bearing, including the following steps:

establishing a tumor-bearing mouse model, conducting administering with non-peripheral quaternary ammonium-modified zinc phthalocyanine and a PD-L1 antibody, followed by laser irradiation.

Preferably, the tumor-bearing mouse may be a bilateral tumor-bearing mouse of melanoma cells B16-F10.

Preferably, the laser irradiation may be conducted 8 h to 12 h after the administration.

Preferably, the laser irradiation may be conducted at a wavelength of 685 nm and a power of 15 mW/cm² for 5 min.

Preferably, the non-peripheral quaternary ammonium-modified zinc phthalocyanine may have a concentration of 200 μM; and

the PD-L1 antibody has a dosage of 50 μg/mouse,

Beneficial effects and outstanding advantages of the present disclosure are as follows:

(1) In the present disclosure, the non-peripheral quaternary ammonium-modified zinc phthalocyanine has a maximum absorption wavelength at 679 nm in an aqueous solution, and a high molar absorption coefficient (up to 105 orders of magnitude); meanwhile, the zinc phthalocyanine has spectral properties not only greatly superior to first-generation photosensitizers, but also superior to other phthalocyanine complexes under clinical trials. For example, the zinc phthalocyanine complex provided by the present disclosure has a maximum absorption wavelength that is red-shifted by 4 nm relative to Pc4 in the United States, that is, a therapeutic spectrum can be red-shifted by 4 nm. Therefore, a tissue penetration ability of therapeutic light is further improved, which is extremely beneficial for PDT and photodynamic diagnosis.

(2) In the present disclosure, the phthalocyanine complex has a clear structure and no positional isomers. The chemical modification of a phthalocyanine parent structure is achieved by introducing monosubstituted groups at non-peripheral positions of the phthalocyanine. Therefore, the target compound has a clear structure, no isomers, and easy preparation.

(3) In the present disclosure, the non-peripheral quaternary ammonium-modified zinc phthalocyanine includes quaternary ammonium groups, such that the compound has excellent amphiphilicity and desirable photodynamic anticancer activity. The zinc phthalocyanine has a photodynamic activity on human cervical cancer cells Hela significantly higher than that of other similar compounds, such as pericyclic tetra-substituted zinc phthalocyanine, 1,8(11),15(18),22(25)-tetrakis(6,8-disulfo-2-naphthyloxy)zinc phthalocyanine octasodium.

(4) In the present disclosure, the non-peripheral quaternary ammonium-modified zinc phthalocyanine has highly efficient synergistic immunotherapy and anti-distal tumor effects. For example, when combined with an immune checkpoint blocker PD-L1 antibody, the zinc phthalocyanine can completely remove in situ tumors, has a 90% inhibitory rate on distal tumors (unilluminated tumors), and can activate tumor immune memory to prevent tumor recurrence. Through a large number of screening experiments, it is found that the non-peripheral quaternary ammonium-modified zinc phthalocyanine combined with the immune checkpoint blocker PD-L1 antibody has a higher ability to inhibit distal tumors than other phthalocyanine photosensitizers, including the precursor 1-[4-(aminoethyl)phenoxy] zinc phthalocyanine and corresponding tetrasubstituted zinc phthalocyanine thereof, further including the 1,8(11),15(18),22(25)-tetrakis(6,8-disulfo-2-naphthyloxy)zinc phthalocyanine octasodium, 1-(6,8-disulfo-2-naphthyloxy) zinc phthalocyanine disodium, tetrasulfo-substituted phthalocyanine, and monosulfo-substituted phthalocyanine.

### DETAILED DESCRIPTION OF THE EMBODIMENTS

The present disclosure is further described below with reference to the accompanying drawings and examples.

### Example 1

A non-peripheral quaternary ammonium-modified zinc phthalocyanine (1-[4-(N,N,N-trimethyl-2-aminoethyl)phenoxy] zinc phthalocyanine iodide) had a structure shown in the following formula:

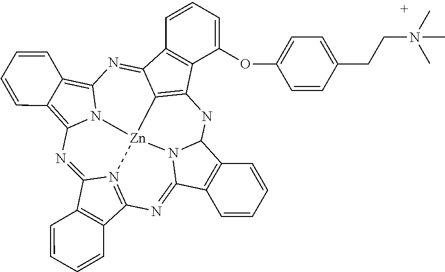

20 mg of 1-[4-(aminoethyl)phenoxy] zinc phthalocyanine (28.5 μmol) and K₂CO₃ (168.28 μmol) were dissolved in a single-neck round-bottomed flask containing 10 mL of anhydrous DMF by ultrasonication; a mixture was cooled to 0° C., added with 2,000 mg of CH₃I slowly, stirred for 30 min, and reacted at room temperature. TLC spot plate was conducted, and terminated after 24 h, a reaction solvent was spin-dried, and a reactant was dissolved with 5 mL of DMF and filtered with a 0.22 μm syringe filter membrane to remove insoluble matters. The solvent was spin-dried by vacuumizing, a remaining product was dissolved with 1 mL of the DMF, passed through an S-X1 gel column, and blue-green components at the forefront were collected using the DMF as an eluent. The solvent was spin-dried by vacuumizing, an obtained product was dissolved in EA and passed through a silica gel column of 100 mesh to 200 mesh (an eluent was EA: DMF=100:1) to remove a yellow component at the forefront, and blue-green bands were collected using EA: DMF=10:1. The solvent was spin-dried by vacuumizing, a remaining product was passed through the S-X1 gel column (an eluent was DMF), and a blue component was collected. The blue component was precipitated in a large amount of solution including n-hexane: DCM=2:1, and dried in an oven at 45° C. to obtain a blue-green solid. The blue-green solid weighed 9.8 mg had a yield of 39.8%. The product had a maximum absorption peak at 674 nm in the DMF and a maximum absorption wavelength at 679 nm in the aqueous solution.

A structural characterization data of the product are as follows: ¹H NMR (400 MHz, DMSO) δ 9.23 (d, J=23.3 Hz, 6H), 8.83 (s, 1H), 8.16 (s, 6H), 7.77 (d, J=6.2 Hz, 1H), 7.44 (s, 2H), 7.37 (s, 2H), 7.09 (s, 1H), 3.90 (s, 2H), 2.74 (s, 2H), 1.50 (s, 2H), 1.26 (s, 4H), 0.84 (s, 3H). HRMS (ESI) m/z calcd for C₄₃H₃₂N₉OZₙ [M-I]⁺: 754.2016; found: 754.2042. HPLC (674 nm): >95%.

### Example 2

The reaction solvent of Example 1 was replaced with 6 mL or 60 mL of anhydrous DMF, other conditions remained unchanged, and a target product could also be obtained. A structural characterization data of the product are as follows: ¹H NMR (400 MHz, DMSO) δ 9.23 (d, J=23.3 Hz, 6H), 8.83 (s, 1H), 8.16 (s, 6H), 7.77 (d, J=6.2 Hz, 1H), 7.44 (s, 2H), 7.37 (s, 2H), 7.09 (s, 1H), 3.90 (s, 2H), 2.74 (s, 2H), 1.50 (s, 2H), 1.26 (s, 4H), 0.84 (s, 3H). HRMS (ESI) m/z calcd for C₄₃H₃₂N₉OZₙ [M-I]⁺: 754.2016; found: 754.2042. HPLC (674 nm): >95%.

### Example 3

2,000 mg of the CH₃I in Example 1 was replaced with 1,000 mg of the CH₃I or 4,000 mg of the CH₃I, other conditions remained unchanged, and a target product could also be obtained. A structural characterization data of the product are as follows: ¹H NMR (400 MHz, DMSO) δ 9.23 (d, J=23.3 Hz, 6H), 8.83 (s, 1H), 8.16 (s, 6H), 7.77 (d, J=6.2 Hz, 1H), 7.44 (s, 2H), 7.37 (s, 2H), 7.09 (s, 1H), 3.90 (s, 2H), 2.74 (s, 2H), 1.50 (s, 2H), 1.26 (s, 4H), 0.84 (s, 3H). HRMS (ESI) m/z calcd for C₄₃H₃₂N₉OZₙ [M-I]⁺: 754.2016; found: 754.2042. HPLC (674 nm): >95%.

### Example 4

The reaction time of Example 1 was changed to 5 h or 50 h, other conditions remained unchanged, and a target product could also be obtained. A structural characterization data of the product are as follows: ¹H NMR (400 MHz, DMSO) δ 9.23 (d, J=23.3 Hz, 6H), 8.83 (s, 1H), 8.16 (s, 6H), 7.77 (d, J=6.2 Hz, 1H), 7.44 (s, 2H), 7.37 (s, 2H), 7.09 (s, 1H), 3.90 (s, 2H), 2.74 (s, 2H), 1.50 (s, 2H), 1.26 (s, 4H), 0.84 (s, 3H). HRMS (ESI) m/z calcd for C₄₃H₃₂N₉OZₙ [M-I]*: 754.2016; found: 754.2042. HPLC (674 nm): >95%.

### Comparative Example 1

1-[4-(aminoethyl)phenoxy] zinc phthalocyanine was synthesized with reference to Chinese patent ZL201711099145.1 (a structure was shown in the following formula).

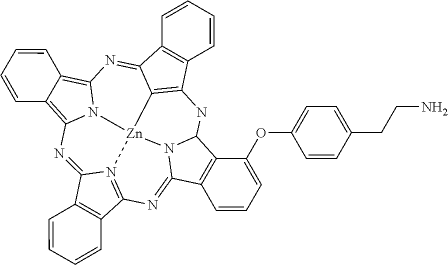

### Comparative Example 2

Other phthalocyanine compounds (structures shown below) were synthesized with reference to published papers (Bioorg. Med. Chem. Lett. 2015, 25: 2386-2389; Chem. Sci., 2018, 9: 2098-2104; Angew. Chem. Int. Ed. 2018, 57: 9885-9890; J. Am. Chem. Soc. 2019, 141: 1366-1372; Theranostics 2019, 9, 6412-6423)

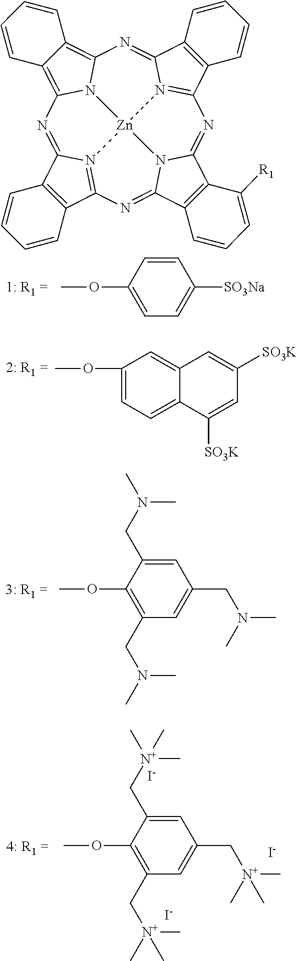

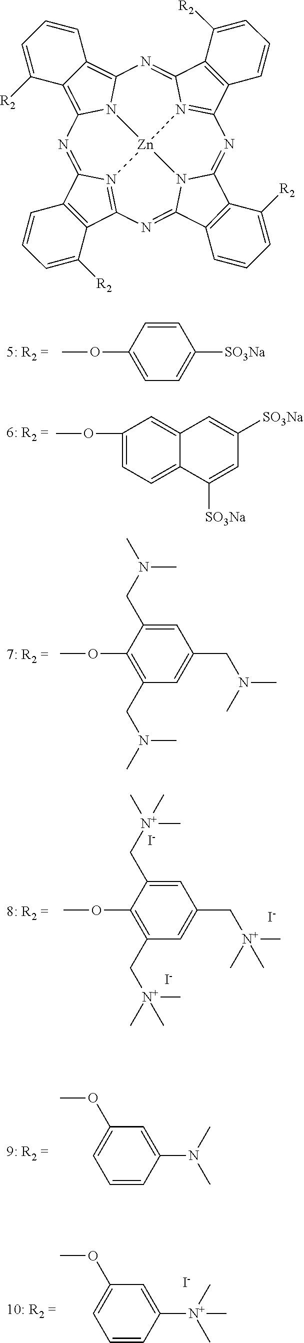

### Example 5

The non-peripheral quaternary ammonium-modified zinc phthalocyanine prepared in Example 1 was dissolved in a 1% castor oil derivative (Cremophor EL, wt %) aqueous solution to prepare a 0.1 mM photosensitizer. The dark toxicity and photodynamic activity of the photosensitizer were tested on human cervical cancer cells Hela.

0.1 mM or 0.2 mM of the photosensitizer was diluted into a cell medium to prepare a cell medium containing zinc phthalocyanine complexes at different concentrations. The cancer cells were cultured in the medium containing different concentrations of zinc phthalocyanine complexes for 2 h; after staining, the medium was discarded; and after washing with PBS, the cells were added into a new medium (without zinc phthalocyanine complexes). Illumination experiment group: the cells were irradiated with red light for 30 min at a power of irradiated light at 15 mW cm⁻² using red light with a wavelength greater than 600 nm as an excitation light source; non-illumination control group: the cells were placed in the dark for 30 min. After illumination or non-illumination, a cell viability was examined by MTT. The specific experimental steps referred to “Bioorganic & Medicinal Chemistry Letters, 2006, 16, 2450-2453”.

The red light with a wavelength greater than 610 nm was provided by a 500 W halogen lamp connected to an insulated water tank and an optical filter greater than 610 nm.

The results show that when being diluted to a concentration of 4 μM (4×10⁻⁶ mol/L), the non-peripheral quaternary ammonium-modified zinc phthalocyanine solution does not kill and inhibit the growth of human cervical cancer cells Hela without irradiation, indicating that the zinc phthalocyanine has no dark toxicity; however, if red light irradiation exists, the zinc phthalocyanine can kill 100% of the cancer cells. By examining a dosage-effect relationship between the concentration of non-peripheral quaternary ammonium-modified zinc phthalocyanine and the cell viability, a half-maximal inhibitory concentration (IC₅₀, a drug concentration required to kill 50% of the cancer cells) under light conditions of 0.9 μM was obtained (the non-peripheral quaternary ammonium-modified zinc phthalocyanine in Example 1). The lower IC₅₀ indicates that the non-peripheral quaternary ammonium-modified zinc phthalocyanine has a relatively high photodynamic activity.

The above 1% castor oil derivative (Cremophor EL, wt %) in water was replaced with 1% castor oil derivative (Cremophor EL, wt %) in PBS or 0.5% castor oil derivative (Cremophor EL, wt %) in water, and same experimental results could also be obtained.

### Example 6

A bilateral tumor-bearing mouse model of melanoma cells B16-F10 (proximal and distal) was established. During PDT, the proximal referred to an illuminated side, and the distal referred to a non-illuminated side. The distribution and metabolism of photosensitizers in the tumor-bearing mouse were investigated by a small animal fluorescence imager and a tissue extraction method, and PDT was conducted under optimal conditions. The following 5 groups of experiments were set up, with 5 mice in each group: a PBS control group; an anti-PD-L1 antibody treatment group; a simple phthalocyanine (no illumination) treatment group; a phthalocyanine+illumination treatment group, namely a PDT group; and a phthalocyanine+illumination+anti-PD-L1 antibody treatment group, namely a combination therapy group. The anti-PD-L1 antibody was purchased from BioX cell.

In the combination therapy group, the PDT group and the simple photosensitizer group, 100 μL of a phthalocyanine compound (at a concentration of 200 μM, diluted with 0.5% CEL) was administered to each mouse. The combination therapy group and the PDT group were irradiated by a laser with a wavelength of 685 nm (at an irradiation power of 15 mW/cm² for 5 min) on the right tumor (proximal tumor) 8 h to 12 h after the administration. The combination therapy group was administered at 50 μg of PD-L1 antibody/mouse immediately after laser treatment, and the antibody group was administered 50 μg of PD-L1 antibody/mouse at the same time. One treatment was given on the first and fourth day separately.

From the first treatment, a body weight and a tumor volume of all mice were measured every other day, and a tumor size was calculated according to a formula: tumor volume=tumor length×width×height×π/6; after 14 consecutive days of observation, a tumor inhibition rate of each experimental group was calculated.

The experimental results show that for the PDT group (phthalocyanine+illumination treatment group), the non-peripheral quaternary ammonium-modified zinc phthalocyanine in Example 1, the non-peripheral amine-modified zinc phthalocyanine in Comparative Example 1, and the other phthalocyanine photosensitizers in Comparative Example 2 have tumor inhibition rates of 51%, 69%, and 50% to 65% on proximal tumors (illuminated tumors) in the B16-F10 tumor-bearing mice, respectively. However, these three groups have almost no inhibitory effect (the tumor inhibition rate was less than 2.5%) on distal tumors (unilluminated tumors), indicating that the photodynamic therapy using phthalocyanine alone cannot inhibit tumor metastasis and metastatic tumors. On the other hand, the PD-L1 antibody therapy alone has a very limited inhibitory effect on proximal and distal tumors, with tumor inhibition rates of 12% and 13%, respectively. Although a photodynamic tumor-inhibitory effect of the zinc phthalocyanine in Example 1 alone is not particularly prominent, the zinc phthalocyanine exhibits an unexpected and significant ability to inhibit distal tumors in synergy with the PD-L1 antibody. In Example 1, the zinc phthalocyanine+illumination+anti-PD-L1 antibody treatment group has a tumor inhibition rate of up to 90% on the distal tumors (unilluminated tumors) of B16-F10 tumor-bearing mice, which was significantly higher than that of the phthalocyanine photosensitizers in Comparative Examples 1 to 2 (under same conditions, the combination anti-PD-L1 antibody treatment has a tumor inhibition rate on distal tumors of 40% to 70%).

More importantly, the mice treated with a combination of the zinc phthalocyanine in Example 1 and the PD-L1 antibody have an immune memory effect, which can effectively prevent tumor recurrence. On a 7th day after the combination therapy of zinc phthalocyanine in Example 1 and PD-L1 antibody, 1×10⁶ B16-F10 cells were subcutaneously injected into a left ventral side of each ICR mouse (5 mice in each group), and the mice were continued to be observed for 21 d. The studies have found that in the combination therapy group of zinc phthalocyanine in Example 1 and PD-L1 antibody, only one of the five mice has developed tumors again, indicating an effective rate of 80% in preventing recurrence. However, the mice in remaining control groups are observed to have obvious melanoma regrowth, and no ability is observed to prevent tumor recurrence. It can be seen that compared with other phthalocyanine photosensitizers, the combination of phthalocyanine-mediated PDT in Example 1 and PD-L1 antibody has significantly enhanced the duration of immune response, successfully stimulated the host immune system, promoted immune memory, and inhibited tumor recurrence.

The above description of examples is merely provided to help illustrate the method of the present disclosure and a core idea thereof. It should be noted that several improvements and modifications may be made by persons of ordinary skill in the art without departing from the principle of the present disclosure, and these improvements and modifications should also fall within the protection scope of the present disclosure. Various amendments to these embodiments are apparent to those of professional skill in the art, and the general principles defined herein may be implemented in other embodiments without departing from the spirit or scope of the present disclosure. Thus, the present disclosure is not limited to the examples shown herein but falls within the widest scope consistent with the principles and novel features disclosed herein.

1. A non-peripheral quaternary ammonium-modified zinc phthalocyanine, having a structural formula shown as follows:

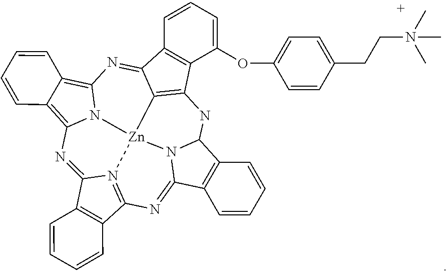

2. A preparation method of the non-peripheral quaternary ammonium-modified zinc phthalocyanine according to claim 1, comprising the following steps: conducting a reaction on 1-[4-(aminoethyl)phenoxy] zinc phthalocyanine and methyl iodide as reactants in N,N-dimethylformamide as a solvent under the protection of nitrogen at 0° C. to a room temperature for 5 h to 50 h; and removing impurities by solvent cleaning and column chromatography isolation to obtain 1-[4-(N,N,N-trimethyl-2-aminoethyl)phenoxy] zinc phthalocyanine iodide.

3. The preparation method according to claim 2, wherein the 1-[4-(aminoethyl)phenoxy] zinc phthalocyanine and the methyl iodide have a mass ratio of 1:(50-200).

4. The preparation method according to claim 2, wherein 1 mg of the 1-[4-(aminoethyl)phenoxy] zinc phthalocyanine requires 0.3 mL to 3 mL of the N,N-dimethylformamide.

5. A preparation method of a photodynamic drug or a photosensitizer comprising using the non-peripheral quaternary ammonium-modified zinc phthalocyanine according to claim 1.

6. The preparation method according to claim 5, wherein the non-peripheral quaternary ammonium-modified zinc phthalocyanine is capable of inhibiting a distal tumor in combination with an immune checkpoint blocker PD-L1 antibody.

7. The preparation method according to claim 5, wherein the photosensitizer or the photodynamic drug prepared by the non-peripheral quaternary ammonium-modified zinc phthalocyanine is capable of treating a tumor in combination with an immune checkpoint blocker.

8. The preparation method according to claim 5, wherein the photodynamic drug or the photosensitizer is used for a photodynamic therapy (PDT), photodynamic diagnosis, or photodynamic disinfection.

9. The preparation method according to claim 8, wherein the PDT comprises a PDT of a malignant tumor, a PDT of a benign tumor, an in vitro photodynamic purification therapy of leukemia, or a PDT of a non-cancer disease.

10. The preparation method according to claim 9, wherein the non-cancer disease comprises a bacterial infection, an oral disease, a macular degeneration-caused eye disease, arteriosclerosis, a traumatic infection, a skin disease, or a viral infection.

11. The preparation method according to claim 8, wherein the photodynamic disinfection comprises photodynamic sterilization and purification of blood or a blood derivative, photodynamic sterilization and disinfection of water, and photodynamic disinfection of a medical or household appliance.

12-20. (canceled)

21. The preparation method according to claim 7, wherein the photodynamic drug or the photosensitizer is used for a photodynamic therapy (PDT), photodynamic diagnosis, or photodynamic disinfection.

22. The preparation method according to claim 21, wherein the PDT comprises a PDT of a malignant tumor, a PDT of a benign tumor, an in vitro photodynamic purification therapy of leukemia, or a PDT of a non-cancer disease.

23. The preparation method according to claim 22, wherein the non-cancer disease comprises a bacterial infection, an oral disease, a macular degeneration-caused eye disease, arteriosclerosis, a traumatic infection, a skin disease, or a viral infection.

24. The preparation method according to claim 21, wherein the photodynamic disinfection comprises photodynamic sterilization and purification of blood or a blood derivative, photodynamic sterilization and disinfection of water, and photodynamic disinfection of a medical or household appliance.
